(19) United States
(12) Patent Application Publication
(10) Pub. No.: US 2012/0107452 A1
(43) Pub. Date: May 3, 2012

(54) AERATION SYSTEMS FOR HORIZONTAL PHOTOBIOREACTORS

(76) Inventors: Andreas Meiser, Puttlingen (DE); Lawrence A. Walmsley, Astoria, NY (US)

(21) Appl. No.: 13/264,396

(22) PCT Filed: Apr. 16, 2010
(86) PCT No.: PCT/US10/31401

§ 371 Date: Jan. 4, 2012

(30) Foreign Application Priority Data
Apr. 16, 2009 (DE) 10 2009 017 628.4

(51) Int. Cl. C12N 1/12; C12N 5/04; C12N 1/20; C12P 1/00; A23J 1/00; C12P 21/00; B01F 3/04; A23L 1/30; A23K 1/18; C12M 1/42; C12P 1/04

(52) U.S. Cl. 426/61; 435/292.1; 435/420; 435/252.1; 435/257.1; 435/41; 435/170; 435/71.2; 435/71.1; 435/70.1; 261/101

(57) ABSTRACT

This invention relates to an aeration system comprising an aeration section, a degassing section and optionally an additional section. This invention also relates to a horizontal photobioreactor comprising the aeration system. This invention further relates to methods of using the photobioreactors.

### CROSS REFERENCE TO RELATED APPLICATION

This application claims the benefit of German Application 102009017628,4, filed Apr. 16, 2009, the entire contents of which are incorporated by reference.

### FIELD OF THE INVENTION

This invention relates to an aeration system comprising an aeration section, a degassing section and optionally an additional section. This invention also relates to a horizontal photobioreactor comprising the aeration system. This invention further relates to methods of using the photobioreactors.

### BACKGROUND OF THE INVENTION

The majority of today's algae production originates from open systems (e.g., “open ponds”). These open systems are susceptible to contamination by foreign algae or parasites. Thus, only algae with very specific growth characteristics can be cultivated in open systems. For example, the species Dunaliella grows in extremely salty water, in which hardly any other organisms can grow, and as such, they can be cultivated in an open system. In addition to being susceptible to contamination, open systems have low productivity. This results in a high production cost of algae. Depending on the area of application, the production costs may be too high and not economical. For example, the production costs in the energy sector are too high to be profitable.

As an alternative to open systems, a large number of dosed systems (“photobioreactors”) have been developed. These include horizontal, flat photobioreactors, tubular photobioreactors and vertical, flat photobioreactors. Many of these photobioreactors are less susceptible to contamination and can reach higher productivities than open systems. However, nearly all of the known photobioreactors have investment costs that are too high to achieve an economical production of algae biomass.

One example of a horizontal photobioreactor is a horizontal film reactor. See, e.g., JP9001182 and WO 2008/079724. In general, these horizontal film reactors have low investment cost, low susceptibility to contamination and good productivities. The low investment cost is due, in part, to the fact that these reactors can be manufactured using a low cost plastic film, e.g., polyethylene (“PE”). PE films can be processed easily by heat welding. See, e.g., WO 2008/079724. The resulting structure is flexible, which can facilitate the mixing of the system. See, e.g., JP9001182.

While the flexibility of the horizontal reactor gives rise to a number of advantages, it also creates some challenges. A major challenge is aeration within the reactor. As used herein, “aeration” refers to a process by which at suspension containing photosynthetic or mixotrophic organisms is enriched with a gas, such as CO₂, and excess oxygen is removed. In photosynthesis, CO₂ is consumed and oxygen is produced.

In general, there are three known methods of aeration. The first method is through external aeration, e.g., by an airlift pump. See, e.g., Acién Fernández et al., “Airlift-driven external-loop tubular photobioreactors for outdoor production of microalgae: assessment of design and performance,” Chemical Engineering Science 56 (2001) and U.S. Pat. No. 4,868,123. Photobioreactors using airlift pumps spatially separate biomass production photosynthetic activity) from the airlift. In the airlift photobioreactor, one compartment is for photosynthetic activity and another is for aeration, degassing and mixing the algae suspension. The airlift and the photosynthesis compartment are connected to each other by tubes or hoses and thus, do not have an integrated design. The airlift can be connected to different reactor types, such as panels and tubes.

In general, airlift photobioreactors achieve a good mass transfer of CO₂ and oxygen between the gaseous and liquid phases. However, external aeration is not practical for mass production of algae. When used on a large scale with large volumes of algae suspension, the process becomes extremely energy consuming and costly. This is because large volumes of algae suspension must be continuously removed from the reactor and, following aeration, the suspension must be returned to the reactor. Additionally, when used on a large scale, multiple airlift pumps are needed, further increasing the costs. Further, when an airlift pump is used in a tubular photobioreactor, the size of the photobioreactor is limited by the length of the tube for photosynthetic activity. Since the amount of oxygen increases over the length of the tube and the amount CO₂ decreases, the maximum length of a tube in a tubular reactor is about 80 m. If the tubes are replaced by panels (horizontal, laminar reactors), it is possible to use a larger volume of algae suspension. However, it becomes difficult to obtain the requisite flow rate at all locations within the reactor.

The second method for aeration is over a semi-permeable diaphragm. Such diaphragms would be too costly for using in a photobioreactor. Moreover, while semi-permeable diaphragms can be used to supply CO₂ to the algae suspension, it is unclear how they would remove oxygen from it.

The third method for aeration is through internal aeration. The internal aerator supplies gaseous CO₂ either through diffusion or injection. Aeration by both supports the removal of oxygen. In addition, because the reactor is aerated throughout, the flow rates of liquids do not need to be high. Also, because the aeration system can be integrated into the photobioreactor structure during manufacture, manufacturing costs can be minimized.

One challenge that has emerged with the use of internal aerators in horizontal, flexible photobioreactors is that a “gas cushion” may form in the reactor between the water surface and the upper reactor wall after a gas or gas mixture is introduced. A gas cushion can form anywhere in the reactor when gas escapes from the algae suspension. When a gas cushion forms, it is above the water surface. Contact with a strong wind could move the reactor. Depending on the wind force, size of the gas cushion, material from which the photobioreactor is manufactured and other factors, the function of the reactor may be substantially impaired and even irreversibly damaged. Gas cushions can also affect the horizontal distribution of the algae suspension. In such a case, the algae may agglomerate, thereby reducing the photosynthetic efficiency of the reactor. Further impairments might occur, e.g., an inefficient, mixing could further reduce the photosynthetic rate or lead to the settling of the biomass or cause other negative effects.

Another challenge relates to the transport of CO₂ and oxygen between the gaseous and liquid phases. To ensure a good mass transfer of CO₂, and potentially oxygen, small gas bubbles are typically blown in at the bottom of the reactor. These bubbles rise and in so doing, an intensive mass transfer takes place between the bubbles and the surrounding algae suspension. Because the reactor is arranged horizontally, the thickness of the algae suspension (measured perpendicular to the water surface) is relatively small and typically within the range of 3-30 cm. In such a case, the bubbles have only a short distance to rise, which, in turn, leads to a short time of contact between the gas bubbles and the algae suspension. As a consequence of the short contact time, the mass transfer between the gaseous and liquid phases may not be sufficient.

A third challenge relates to supplying the gas or gas mixture to the algae suspension. A fixed structure, such as PCV pipe, may be used as a gas supply pipe. However, fixed structures increase the manufacturing costs of the reactor system significantly. Alternatively, aeration may be performed by a system of chambers integrated into the reactor. These chambers may be manufactured by welding plastic films onto one of the reactor walls, usually on the lower reactor wall. The chamber system provides vents at defined locations from which the gas can migrate to the algae suspension. Because the chamber system could have a lower density than the surrounding algae suspension, it could possess a higher buoyancy than the algae suspension, causing it to move towards the water surface. The closer the aeration system is to the water surface, the lower the contact time between the as bubbles and the algae suspension.

Accordingly, the present invention provides an aeration system comprising an aeration section, a degassing section and optionally an additional section. The present invention also provides a horizontal photobioreactor comprising the aeration system. The photobioreactors of the present invention can be used in the large scale production of algae biomass. Importantly, the photobioreactors can be manufactured at very low cost, exhibit low operating costs and have low susceptibility to contamination. The present invention also provides methods of using the photobioreactors.

### SUMMARY OF THE INVENTION

The present invention provides an aeration system comprising an aeration section, a degassing section and optionally an additional section. The additional section may be a growth section.

The present invention also provides a horizontal photobioreactor comprising an aeration system. The aeration system comprises an aeration section, a degassing section and optionally an additional section. Each section may be lowered below the other sections, or all the sections may be lowered below the surrounding water body.

The present invention also provides methods of using the horizontal photobioreactors. The horizontal photobioreactors may be used to grow photosynthetic or mixotrophic organisms. Alternatively, the horizontal photobioreactors may be used to produce a biomass, a biofuel or a product selected from biochemicals, amino acids, fine chemicals, nutriceuticals, pharmaceuticals, energy products, protein, feed for cattle, fish and other species, protein source for human nutrition and mineral rich food for human consumption.

### BRIEF DESCRIPTION OF THE FIGURES

FIG. 1 is a top view of a cutout of a horizontal photobioreactor comprising an integrated aeration system. The aeration system comprises an aeration section and a degassing section.

FIG. 2 is a cross section of a cutout of a horizontal photobioreactor comprising an integrated aeration system. The aeration system comprises an aeration section and a degassing section.

FIG. 3 is a three-dimensional view of a cutout of a horizontal photobioreactor composing an integrated aeration system. The aeration system comprises an aeration section and a degassing section.

FIG. 4 is a top view of a cutout of a horizontal photobioreactor comprising an integrated aeration system. The aeration system comprises an aeration section, a degassing section and a growth section.

FIG. 5 is a three-dimensional view of a cutout of a horizontal photobioreactor comprising an integrated aeration system. The aeration system comprises an aeration section, a degassing section and a growth section.

FIG. 6 is a three-dimensional view of a cutout of a horizontal photobioreactor comprising an integrated, lowered aeration section.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials and methods are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

### Aeration Systems

The present invention provides an aeration system comprising an aeration section, a degassing section and optionally an additional section. While these sections are described separately below, it is to be understood that the aeration system of the present invention may comprise any combination of these sections.

The aeration section is designed to facilitate the mass the transfer of CO₂ and oxygen between the supplied gas mixture and the suspension. The aeration section may be any size or shape, depending on the size and shape of the photobioreactor in which it is used, as well as the surrounding water body. The aeration section may have a width in the range of about 2 cm to about 1000 m, preferably about 500 cm to about 200 m. The aeration section may have a length in the range of about 1 m to about 1000 m, preferably about 5 m to about 200 m and even more preferably about 1 m to about 120 m. In some embodiments, the aeration section is rectangular. In some embodiments, the aeration section covers the entire width of the reactor. In other embodiments, the aeration section covers a portion of the width of the reactor.

The aeration section may or may not be integrated into the design of the photobioreactor in which it is used. When the aeration section is not integrated into the design of the photobioreactor, the upper and lower walls of the aeration system are connected in such a way to form the aeration section (and the degassing section).

The upper and lower walls of the aeration system are made from a flexible material. Suitable materials include, but are not limited to, materials made from polyolefins e.g., polyethylene and polypropylene, polyacrylates, polyamides, polycarbonates, water insoluble cellulose esters and polyester films. Preferably, the walls are manufactured from a polyethylene film.

The upper wall of the aeration system may be directly or indirectly connected to the lower wall. The connections between the upper and lower walls prevent or minimize the formation of a large gas cushion between the surface of the suspension and the upper wall. The connections between the upper and lower walls may be formed by any technique known in the art, such as welding.

The connections between the upper and lower walls of the aeration system may be any shape or size. Suitable shapes include, but are not limited to, circular dots (punctual dots), punctiforms, and square dots. When the connections are circular, the diameter may be about 2 mm to about 50 mm, preferably about 4 mm to about 20 mm and even more preferably about $ mm to about 12 mm. When the connections are square, the length may be about 2 mm to about 50 mm, preferably about 4 mm to about 20 mm and even more preferably about 8 mm to about 12 mm. Preferably, the connections are circular with a diameter of about 1 cm.

The connections between the upper and lower walls of the aeration system may be arranged at regular or irregular distances. The connections may be arranged in one or more rows. The distance between the connections in the same row may be about 2 to about 50 cm, preferably about 4 to about 25 cm and even more preferably, about 10 cm. The distance between the rows may be about 2 to about 50 cm, preferably about 4 to about 25 cm and even more preferably, about 10 cm. In some embodiments, the rows run parallel to the external boundary of the aeration system.

Alternatively, the upper and lower walls of the aeration system may be connected indirectly through an additional material. That is, the upper wall may be connected to an additional material, which, in turn, is connected to the lower wall. The additional material may be manufactured from any material known in the art. Suitable materials include, but not limited to, materials made from polyolefins e.g., polyethylene and polypropylene, polyacrylates, polyamides, polycarbonates, water insoluble cellulose esters and polyester films.

When the aeration section is integrated into the photobioreactor M which it is used, the upper and lower walls of the photobioreactor are connected in such a way to form the aeration section (and the degassing section). The upper reactor wall may be directly or indirectly connected to the lower reactor wall. The connections between the upper and lower reactor walls prevent or initialize the formation of a large gas cushion between the surface of the suspension and the upper reactor wall. The connections between the upper and lower reactor walls may be formed by any technique known in the art, such as welding.

The connections between the upper and lower reactor walls may be any shape or size. Suitable shapes include: but are not limited to, circular dots (punctual dots), punctiforms, and square dots. When the connections are circular, the diameter may be about 2 mm to about 50 mm, preferably about 4 mm to about 20 mm and even more preferably about 8 mm to about 12 mm. When the connections are square, the length may be about 2 mm to about 50 min preferably about 4 mm to about 20 mm and even more preferably about 8 mm to about 12 mm. Preferably, the connections are circular with a diameter of about 1 cm.

The connections between the upper and lower reactor walls may be arranged at regular or irregular distances. The connections may be arranged in one or more rows. The distance between the connections in the same row may be about 2 cm to about 50 cm, preferably about 4 cm to about 25 cm and even more preferably, about 10 cm. The distance between the rows may be about 2 cm to about 50 cm, preferably about 4 cm to about 25 cm and even more preferably, about 10 cm. In some embodiments, the rows run parallel to the external boundary of the reactor.

Alternatively, the upper and lower reactor walls may be connected indirectly through an additional material. That is, the upper reactor wall may be connected to an additional material, which, in turn, is connected to the lower reactor wall. The additional material may be manufactured from any material known in the art. Suitable materials include, but not limited to, materials made from polyolefins e.g., polyethylene and polypropylene, polyacrylates, polyamides, polycarbonates, water insoluble cellulose esters and polyester films.

The aeration section may be lowered, in whole or in part, into the surrounding water body. Lowering the aeration section or a part of it increases the time of contact between the gas mixture and the suspension. The amount that the aeration section is lowered depends on the desired level of aeration and the type of surrounding body. In general, the level of aeration increases with the depth of the aeration section in the surrounding water body. Also, the deeper the aeration section, the deeper the surrounding water body must be. The aeration section may be lowered to about 15 cm to about 10 m below the surface of the water body. Preferably, the aeration section is about 40 cm below the surface.

The aeration section may be lowered by manufacturing the reactor wall using a material with high density. Alternatively, the aeration section may be lowered by increasing the amount of material in the reactor wall. In addition, the aeration section may be lowered by fastening it to an object outside of the reactor, for example, by fastening it to the bottom of the water body. Also, the aeration section may be lowered by adding a material having a higher density than the surrounding water body. Additionally, the aeration section may be lowered by manufacturing it such that it contains one or more chambers that can be filled with a material having a higher density than the surrounding water body. Such materials include, but are not limited to, salt-in-water solution and sand. In preferred embodiments, the aeration section comprises a chamber filled with a salt-in-water solution or it is fastened to an outside object.

When the aeration section is lowered using a material with high density, both external walls of the aeration section are formed from the lower reactor wall. A film is placed between these two external walls and fixed on the material with high density. The connection is realized by punctual dots at regular or irregular distances. The distance between the dots may be about 2 cm to about 30 cm. Preferably, the distance between the dots is about 10 cm. The film is connected at its upper end to the upper reactor wall directly by techniques known in the art, e.g., welding.

The aeration section may optionally comprise films, flaps or other objects attached to it to affect the currents.

In addition to the aeration section, the aeration systems of the present invention comprise a degassing section. The degassing section is designed to reduce the gas bubbles in the suspension after passing through, or when staying within, it. The gas bubbles migrate from the liquid to the gaseous phase above. The degassing section may be used to collect the gas mixture accumulating over the water surface and channel it out of the photobioreactor. The degassing section may be separated by one or more devices from other sections of the reactor to prevent shifting of gas bubbles into other sections, thereby minimizing the formation of gas cushions.

The degassing section is located next to the aeration section. When the aeration system comprises a growth section, the degassing section is located on one side of the growth section and the aeration section is on the other. When the aeration system comprises a lowered aeration section, the degassing section sans above the center of the aeration section.

The size and shape of the degassing section may be varied, depending on the size and shape of the photobioreactor in which it is used, as well as the surrounding water body. The degassing section may have a width in the range of about 5 cm to about 80 cm, preferably about 10 cm to about 40 cm and even more preferably about 15 cm to about 30 cm. The degassing section may have a length in the range of about 1 m to about 1000 m, preferably about 5 in to about 200 m and even more preferably about 8 m to about 120 m. In preferred embodiments, the degassing section has a width of about 20 cm and a length of about 10 m.

The degassing section may or may not be integrated into the design of the photobioreactor. When the degassing section is not integrated into the photobioreactor, the upper and lower walls of the aeration system are connected in such a way to form the degassing section (and the aeration section).

When the degassing section is integrated into the photobioreactor in which it is used, the upper and lower walls of the photobioreactor are connected in such a way to form the degassing section (and the aeration section), in the degassing section, the upper and lower reactor walls are completely separated from each other, except at: (1) the connections outside boundary of the reactor; (2) the connections at the passage to the growth section, if present (discussed below); and (3) the connections dose to an added film, if present (discussed below).

The degassing section collects the as mixture leaving the liquid phase. The degassing section may be constructed to remove the collected gas mixture at one of its ends. The degassing section may comprise connections to the periphery to remove excess gas. In some embodiments, a hose is fastened to the end of degassing section to lead the gas mixture out of the reactor. When the aeration system or the photobioreactor in which it is used contains multiple degassing sections, two or more of them may be connected by a tubular chamber to reduce the number of connections to periphery.

The degassing section may be lowered, in whole or in part, into the surrounding water body. The degassing section may be lowered to about 15 cm to about 10 m below the surface of the water body. Preferably, the degassing section is about 40 cm below the surface.

The degassing section may be lowered by manufacturing the reactor wall using a material with high density. Alternatively, the degassing section may be lowered by increasing the amount of material in the reactor wall. In addition, the degassing section may be lowered by listening it to an object outside of the reactor, for example, by fastening it to the bottom of the water body. Also, the degassing section may be lowered by adding a material having a higher density than the surrounding water body. Additionally, the degassing section may be lowered by manufacturing it such that it contains one or more chambers that can be filled with a material having a higher density than the surrounding water body. Such materials include, but are not limited to, salt-M-water solution and sand. In preferred embodiments, the degassing section comprises a chamber tilled with a salt-in-water solution or it is fastened to an outside object.

The aeration systems of the present invention may optionally comprise additional sections. One such additional section is a growth section. In the growth section, the biomass consumes additional CO₂ and undergoes photosynthesis. After the CO₂ is depleted, in part or in whole, the suspension is returned to the aeration section or the degassing section.

In embodiments in which the aeration system comprises an aeration section, a degassing section and a growth section, a plastic film is welded in the aeration system, where the aeration section and the growth section meet. This film is welded over its entire length with the upper wall of the aeration system such that the weld seam stretches over the entire width of the aeration system. The film is connected to the lower wall of the aeration system through connections. These connections may be airy shape or size. Suitable shapes include, but are not limited to, circular dots (punctual dots), punctiforms, and square dots. When the connections are circular, the diameter may be about 2 mm to about 50 mm, preferably about 4 mm to about 20 mm and even more preferably about 8 mm to about 12 mm. When the connections are square, the length may be about 2 mm to about 50 mm, preferably about 4 mm to about 20 mm and even more preferably about 8 mm to about 12 mm. Preferably, the connections are circular with a diameter of about 1 cm.

The connections between the film and the lower wall of the aeration system may be arranged at regular or irregular distances. The connections may be arranged in one or more rows. The distance between the connections in the same row may be about 2 cm to about 50 cm, preferably about 3 cm to about 15 cm and even more preferably, about 5 cm. The upper and lower connections preferably are vertically misaligned to each other, i.e., the upper connection is not directly above the lower connections.

In embodiments in which the aeration system comprises a lowered aeration section, a degassing section and a growth section, the upper and lower wall of the aeration system are connected at the transition between the degassing section and the growth section. These connections run along the entire transition zone between the degassing section and the growth section. The connections between the upper and lower walls may be any shape or size. Suitable shapes include, but are not limited to, circular dots (punctual dots), punctiforms, and square dots. When the connections are circular, the diameter may be about 2 mm to about 50 mm, preferably about 4 mm to about 20 mm and even more preferably about 8 mm to about 12 mm. When the connections are square, the length may be about 2 mm to about 50 mm, preferably about 4 mm to about 20 mm and even more preferably about 8 mm to about 12 mm. Preferably, the connections are circular with a diameter of about 1 cm.

The connections between the upper and lower walls of the aeration system may be arranged at regular or irregular distances. The connections may be arranged in one or more rows. The distance between the connections in the same row may be about 2 cm to about 50 cm, preferably about 3 cm to about 15 cm and even more preferably, about 5 cm. The distance between the rows may be about 2 cm to about 50 cm, preferably about: 4 cm to about 25 cm and even more preferably, about 10 cm.

The growth section may be lowered, in whole or in pail, into the surrounding water body. The growth section may be lowered to about 15 cm to about 10 m below the surface of the water body. Preferably, the growth section is about 40 cm below the surface.

The growth section may be lowered by manufacturing the reactor wall using a material with high density. Alternatively, the growth section may be lowered by increasing the amount of material in the reactor wall. In addition, the growth section may be lowered by fastening it to an object outside of the reactor, for example, by fastening it to the bottom of the water body. Also, the growth section may be lowered by adding a material having a higher density than the surrounding water body. Additionally, the growth section may be lowered by manufacturing it such that it contains one or more chambers that can be filled with a material having a higher density than the surrounding water body. Such materials include, but are not limited to, salt-in-water solution and sand. In preferred embodiments, the growth section comprises a chamber filled with a salt, in-water solution or it is fastened to an outside object.

The arrangement of the aeration section, the degassing section and the optional additional sections in the aeration system may be varied. The sections may be repeated in the same or an easily modified pattern several times over the aeration system or the photobioreactor in which they are used. The sections may also be arranged in a circular pattern.

A gas mixture is supplied to the aeration section through parallel chambers. The parallel chambers are integrated into the aeration section. The size and shape of the parallel chambers may be varied, depending on the selected photosynthetic or mixotrophic organism, the climate, the use of the biomass, the pressure of the supplied gas mixture, the reactor volume, and the thickness of the medium (light path). In preferred embodiments, the parallel chambers are tubular or elliptic. The length of the chambers may be about 1 m to 1000 m, preferably about 5 m to about 200 in and even more preferably, about 10 m. The parallel chambers have a diameter of about 0.2 cm to about 10 cm, preferably, about 0.5 cm to about 4 cm, and even more preferably, about 0.8 cm to about 1 cm. The parallel chambers have holes. In some embodiments, the diameter of the hole is about 0.1 mm to about 1.5 mm, preferably about 0.2 mm to about 1 mm, and even more preferably about 0.6 mm. In some embodiments, the holes are placed about 1 cm apart.

The location of the parallel chambers depends on the configuration of the aeration system or the photobioreactor in which the aeration system is used. In embodiments in which the aeration system comprises an aeration section and a degassing section, the parallel chambers are located in the middle between two lines created by the connections of the upper and lower walls of the aeration system. Similarly, in embodiments in which the aeration system comprises an aeration section, a degassing section and a growth section, the parallel chambers are located in the middle between two lines created by the connections of the upper and lower walls of the aeration system. In embodiments in which the aeration system comprises a lowered aeration section, a degassing section and a growth section, the parallel chambers are located near the center of the high density chamber and at one side of the added film.

The parallel chambers are connected at one of the long sides of the aeration system by a long chamber, running perpendicular to it. The long chamber, through connections to the periphery, supplies the parallel chamber with the gas mixture. The dimensions and location of the long: chamber may also be varied, depending on the site of the aeration system, the size of the photobioreactor in which the aeration system is used, the selected photosynthetic or mixotrophic organism, the pressure of the supplied gas mixture, the flow rate, and the size of the parallel chambers. In some embodiments, the long chamber runs over the entire length of the aeration system, preferably the length of the long chamber is about 50 m. The long chamber has width of about 1 to about 20 cm, preferably, about 2 to about 10 cm, and even more preferably, about 2.5 cm. The long chamber may have holes. Preferably, the long chamber does not have any holes.

The parallel chambers and the long chamber are manufactured using materials known in the art, Suitable materials include, but are not limited to, materials made from polyolefins e.g., polyethylene and polypropylene, polyacrylates, polyamides, polycarbonates, water insoluble cellulose esters and polyester films. Preferably, the chambers are manufactured from a polyethylene film. The chambers are manufactured by methods known in the art. Preferably, these chambers are manufactured by welding a polyethylene film onto the lower wall of the aeration system.

The thickness of the chamber walls will vary depending on the material from which they are manufactured. The thickness may be about 50 μm to about 800 μm, preferably about 100 μm to about 400 μm, and even more preferably, about 200 μm. In some embodiments, walls consist of polyethylene film having a thickness of 200 μm.

A preferred aeration system of the present invention comprises an aeration section and a degassing section. The walls of the aeration system are made from a flexible material. Connections between the upper and lower walls form the aeration section and the degassing section.

A preferred aeration system of the present invention comprises an aeration section, a degassing section and a growth section. The walls of the aeration system are made from a flexible material, Connections between the upper and lower walls form the aeration section and the degassing section.

Preferably, in each of the preferred aeration systems, the flexible material is polyethylene.

Preferably, in each of the preferred aeration systems, the aeration section comprises parallel chambers and a long chamber. The chambers supply a gas mixture to the aeration section.

Preferably, in each of the preferred aeration systems, the degassing section comprises connections to the periphery to transport excess gas out of the aeration system.

Preferably, in each of the preferred aeration systems, the aeration system further comprises a photosynthetic or mixotrophic organism and growth medium.

### Photobioreactors

The photobioreactors of the present invention comprise an aeration system as described, in the preceding section. The aeration system may or may not be integrated into the design of the photobioreactor. When the aeration system is not integrated into the photobioreactor, it may be used with any horizontal, flexible photobioreactor known in the art. Suitable photobioreactors include, but are not limited to those disclosed in U.S. Pat. No. 4,868,123 and U.S. Pat. No. 3,955,317. In some embodiments, the photobioreactor is a raft formed of transparent tubes.

When the aeration system is integrated into the photobioreactor, the upper and lower walls of the reactor are connected in such a way to form the aeration system. The external walls of the photobioreactor may be manufactured from a flexible, thin plastic material. Suitable materials include material made from polyolefins, e.g., polyethylene and polypropylene, polyacrylates, polyamides, polycarbonates, water insoluble cellulose esters and polyester films. Preferably, the walls are manufactured from a polyethylene film. The walls may further comprise a material selected from metal, additional plastic, fibers or sand.

When the walls are manufactured from polyethylene film, the connections between the walls may be provided by methods known in the art. In preferred embodiments, the connections are provided by means of welding.

The thickness of the walls will vary depending on the material from which they are manufactured. The thickness may be about 50 μm to about 800 μm. In some embodiments, the walls consist of polyethylene film having a thickness of 200 μm.

The dimensions of the photobioreactor may vary according to the size of the surrounding water body, the biomass production capacity, the reactor material, the manufacturing process, the environmental conditions of the specific site, the selected photosynthetic or mixotrophic organism and other factors known in the art. In some embodiments, the photobioreactor has a length of about 50 m and a width of about 10 m.

Two or more photobioreactors may be connected to each other to form a system. The photobioreactors may be arranged in pairs so that the medium flows in the two reactors in different directions. In one reactor, the medium flows from “right to left” and the other “left to right”. The two reactors are connected to each other at their ends so that the suspension flows through both reactors and a cyclic flow is formed. In addition, multiple photobioreactors may be arranged in a circular pattern, semi-circular pattern, or long rows. Alternatively, two or more photobioreactors may be stacked, one above the other. In addition, one or more of the photobioreactors may be arranged in any of the patterns described above with any other type of photobioreactor. A preferred system of photobioreactors comprises two or more photobioreactors connected to each other, wherein at least one is a photobioreactor of the present invention. Preferably, the photobioreactors are arranged in a circular pattern.

The photobioreactors of the present invention may optionally comprise additional components. Such additional components are known in the art and include, but are not limited to, a peripheral apparatus, e.g., compressors, to supply CO₂ containing gas to the reactor; connections at the reactor, e.g., to harvest biomass or to add nutrients; pumps; filters; temperature control systems; buoyancy control systems; and controls and sensors for monitoring internal and external conditions that might impact or enhance the growth of the photosynthetic or mixotrophic organism.

A preferred photobioreactor of the present invention comprises an aeration system, which, in turn, comprises an aeration section and a degassing section. The walls of the aeration system are made from a flexible material. Connections between the upper and lower walls form the aeration section and the degassing section.

A preferred photobioreactor of the present invention comprises an aeration system, which, in turn, comprises an aeration section, a degassing section and a growth section. The walls of the aeration system are made from a flexible material. Connections between the upper and lower walls form the aeration section and the degassing section.

Preferably, in each of these preferred photobioreactors, the flexible material is polyethylene.

Preferably, in each of these preferred photobioreactors, the aeration section comprises parallel chambers and a long chamber. The chambers supply a gas mixture to the aeration section.

Preferably, in each of these preferred photobioreactors, the degassing section comprises connections to the periphery to transport excess gas out of the aeration system.

Preferably, in each of these preferred photobioreactors, the aeration system further comprises a photosynthetic or mixotrophic organism and growth medium.

A preferred horizontal photobioreactor of the present invention comprises an aeration system, which, in turn, comprises an aeration section and a degassing section. Connections between the upper and lower walls of the photobioreactor form the aeration section and the degassing section.

Preferably, the upper and lower walls of the photobioreactor are directly connected in the aeration section.

Preferably, the horizontal photobioreactor further comprises a growth section. This horizontal photobioreactor may further comprise a film where the aeration section and growth section meet. The film and the lower wall of the photobioreactor are connected.

A preferred horizontal photobioreactor comprises an aeration section and a degassing section. The aeration section is lowered below the degassing section.

A preferred horizontal photobioreactor comprises an aeration section, a degassing section and a growth section. The aeration section is lowered below the degassing section and the growth section. The upper and lower walls of the photobioreactor are connected to form the degassing section and the growth section.

A preferred horizontal photobioreactor comprises an aeration section, a degassing section, and a growth section. The aeration section, degassing section and the growth section are lowered below a surrounding water body.

Preferably, when a section of the photobioreactor is lowered, it is lowered by a method selected from the group consisting of manufacturing the photobioreactor wall using a material with high density, increasing the amount of material in the photobioreactor wall, fastening the section to an object outside of the photobioreactor, adding a material with higher density than a surrounding water body to the section, and manufacturing the section to contain one or more chambers that can be filled with a high density material.

Preferably, in each of the preferred horizontal photobioreactors, the upper and lower reactor walls comprise a flexible material. More preferably, the flexible material is polyethylene.

Preferably, in each of the preferred horizontal photobioreactors, the aeration section comprises parallel chambers and a long chamber. These chambers supply a gas mixture to the aeration section. More preferably, the gas mixture comprises carbon dioxide.

Preferably, in each of the preferred horizontal photobioreactors, the degassing section comprises connections to periphery to transport excess gas out of the aeration system.

Preferably, each of the preferred, horizontal photobioreactors further comprise a photosynthetic or mixotrophic organism and growth medium.

Preferably, in each of the preferred horizontal photobioreactors, the growth section and the degassing section are separated by connections between the upper and lower walls of the photobioreactor.

Preferably, in each of the preferred horizontal photobioreactor the sections are repeated within the photobioreactor.

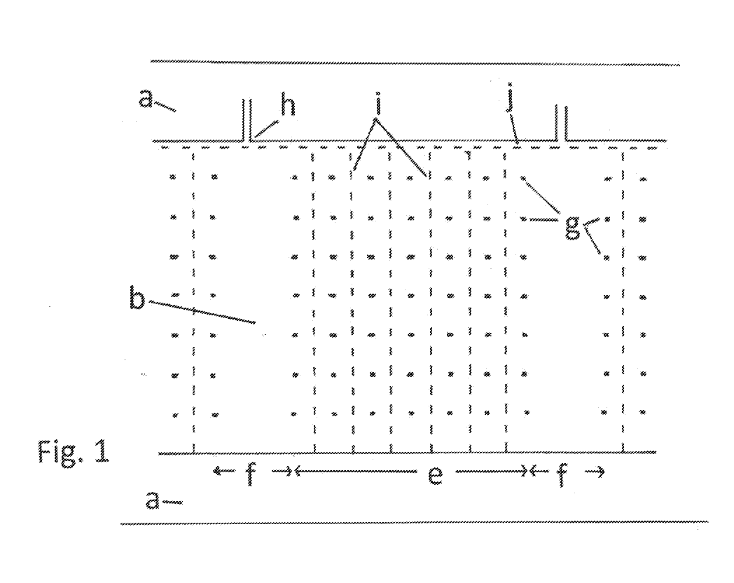

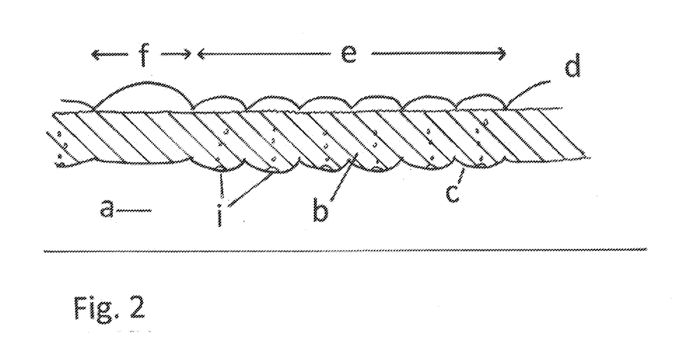

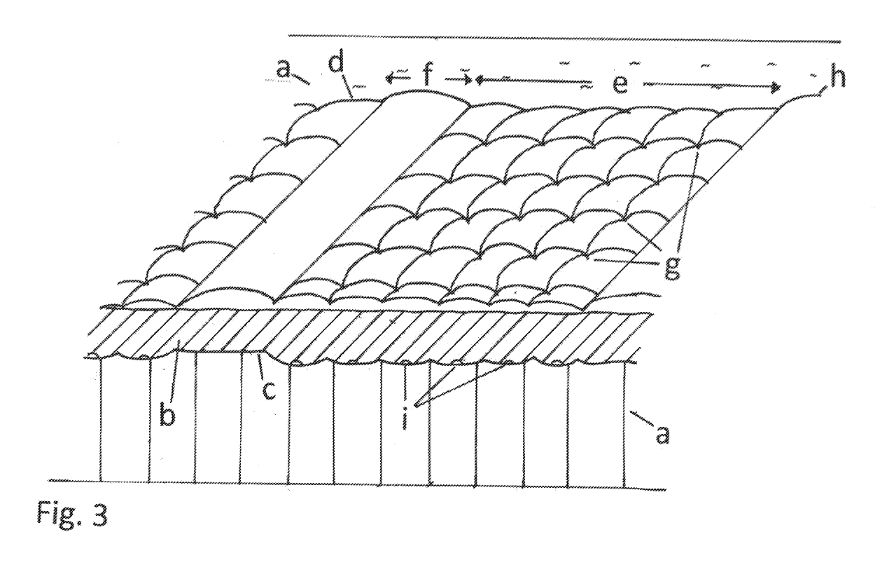

A preferred photobioreactor comprises an integrated aeration system, which, in turn, comprises an aeration section and degassing section. Referring to FIG. 1, FIG. 2 and FIG. 3, the photobioreactor is located in a water body (a) and contains an algae suspension (b) dispersed in a thin, even layer. The photobioreactor is divided structurally into two sections: an aeration section (e) and degassing section (f). In the aeration section, the lower (c) and upper (d) reactor walls are connected by punctual dots (g). The connections (g) are arranged in parallel rows such that the distance between connections in the same row is about 10 cm and the distance between rows is about 10 cm. The connections are manufactured by welding into a circular shape, having a diameter of about 1 cm. In the degassing section (f), the lower (c) and upper (d) reactor walls are completely separated from each other, except thr the connections at the boundary of the reactor. A hose is fastened to one end of the degassing section (h) to lead the collected gas mixture out of the reactor. Tubular parallel chambers (i) are integrated in the aeration section (e) to supply a gas mixture comprising CO₂. When the gas mixture is introduced into the reactor, bubbles rise in the aeration section (e). The connections (g) between the upper and lower reactor wall prevent the inflation of the reactor, and thus, prevent the formation of a large gas cushion. The surplus gas flows into one of the two adjacent degassing sections (f), where it will be led out. Some of the algae suspension in the reactor follows the current of the as mixture at the surface of the aeration section and some flows toward the degassing section. At the bottom, the algae suspension flows from the degassing section into the aeration section, in order to maintain a stable liquid balance.

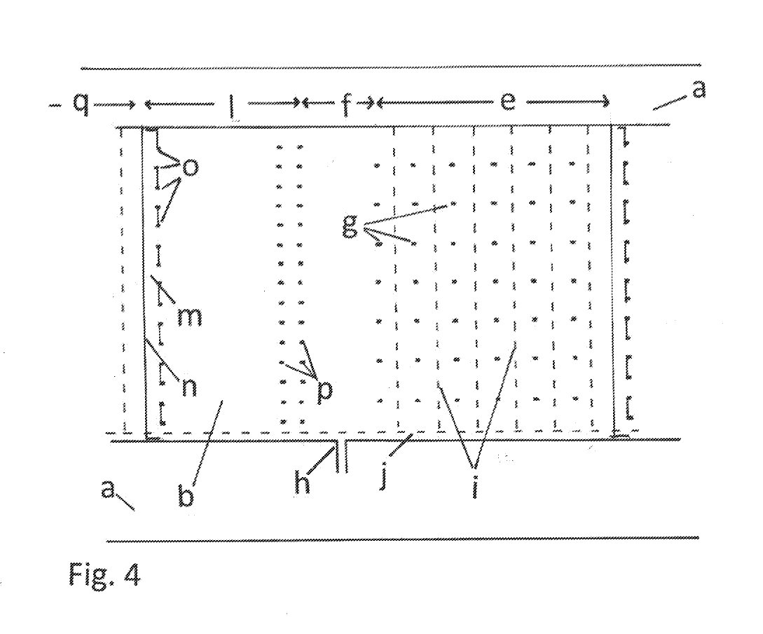

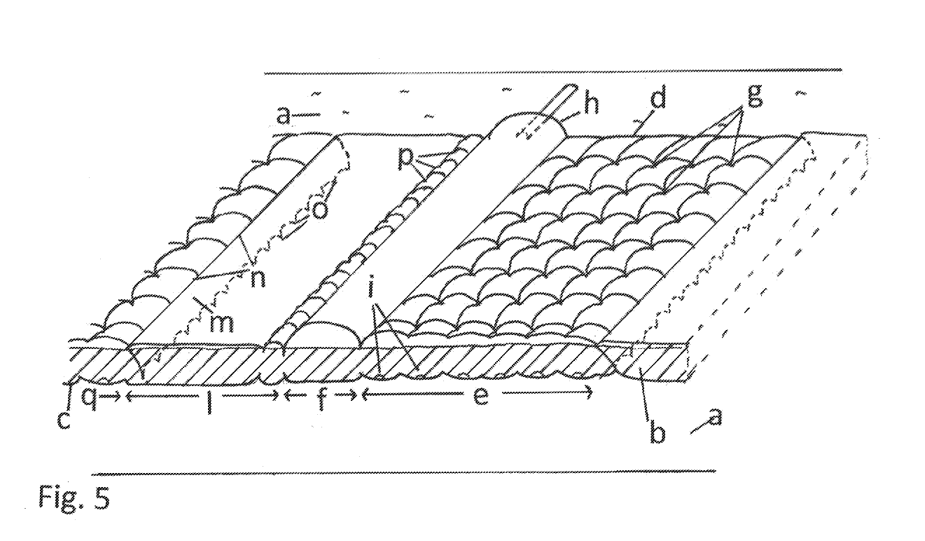

A preferred embodiment is a photobioreactor comprising an integrated aeration system, which, in turn, comprises an aeration section, degassing section and growth section. Referring to FIG. 4 and FIG. 5, the photobioreactor is located in a water body (a) and contains an algae suspension (b) dispersed in a thin, even layer. The photobioreactor is divided structurally into three sections: an aeration section (e), a degassing section (f), and a growth section (l). The three sections alternate.

In the aeration section (e), the lower (c) and upper (d) reactor walls are connected by punctual dots (g). The connections (g) are arranged in parallel rows such that the distance between connections in the same row is about 10 cm and the distance between rows is about 10 cm. The connections are manufactured by welding into a circular shape, having a diameter of about 1 cm. An additional plastic film (m) is welded in the reactor, where the aeration section (e) and the growth section (l) meet. The film (m) is welded over its entire length with the upper reactor wall (d) and is connected to the lower reactor wall (c) through punctual dot connections (o). The punctual connections (o) are about 5 cm from each other. The upper (n) and lower (o) connections are vertically misaligned to each other.

In the degassing section (f), the lower (c) and upper (d) reactor walls are completely separated from each other, except for the connections at the boundary of the reactor. A hose is fastened to one end of the degassing section (h) to lead the collected gas mixture out of the reactor.

The growth section (l) is located between the aeration section (e) and the degassing section (f). The growth section (l) and the aeration section (e) are partially separated by film (m). The growth section (l) and the degassing section (f) are separated by two rows of connections (p) between the upper (d) and lower (c) reactor walls. The distance between the rows and the distance between connections in the same row is about 5 cm.

Tubular parallel chambers (i) are integrated in the aeration section (e) to supply a gas mixture comprising CO₂. Within the aeration section (e), neighboring parallel chambers (i) are located about 10 cm apart. Further, the parallel chambers (i) are located in the middle between two lines created by the connections of the upper (d) and tower (c) reactor walls. All the parallel chambers (i) are connected to a long chamber (i) at one of the long sides of the reactor. Long chamber (j) runs over the entire length of the reactor and is about 50 m in length. Chambers (i, j) are manufactured by welding a PE film onto the lower (c) reactor wall, Parallel chamber (i) has a width of about 1.5 cm and holes that about 1 cm from each other. Long chamber (j) has a width of about 2.5 cm and preferably, no holes.

When the as mixture is introduced into the reactor via parallel chambers (i), gas bubbles rise in the aeration section (e). The connections (g) between the upper and lower reactor wall prevent the inflation of the reactor, and thus, prevent the formation of a large gas cushion. The surplus gas flows into one of the adjacent sections. The film (m) functions as a flap to direct the flow of gas from the aeration section (e) to the degassing section (f), rather than to the adjacent growth section (l). As the gas mixture flows in a single direction, it carries the liquid in the same direction, i.e., from the aeration section to the degassing section.

Both momentum and the flowing medium cause the algae suspension to flow into the growth section (l). As the algae suspension flows into the growth section (l), the connections that partially separate the upper and lower reactor walls in the degassing section prevent the formation of a large gas cushion. The algae suspension flows from the growth section (l) into the next aeration section (q), passing the zone under film (m). At this point, the flows within the reactor repeat as described above.

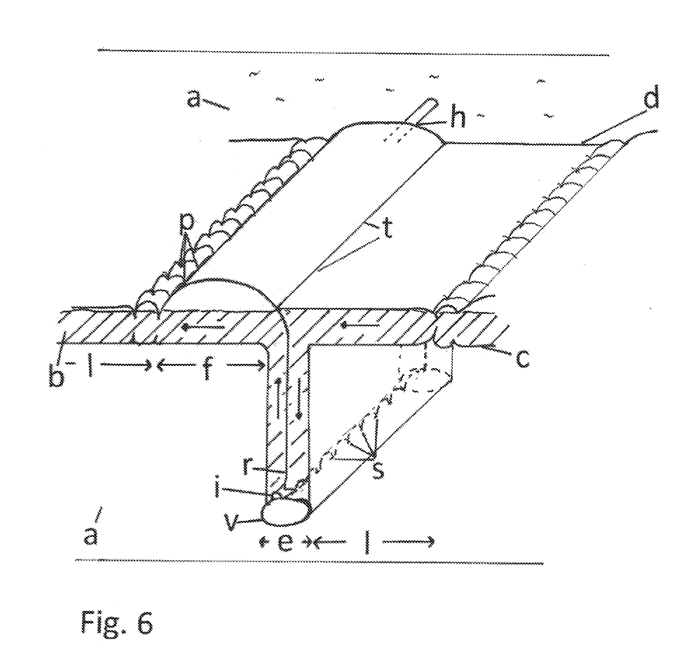

A preferred embodiment is a photobioreactor comprising an integrated aeration system, which, in turn, comprises three sections: an aeration section, degassing section, and growth section, in this preferred photobioreactor, the growth section is horizontally arranged and the aeration section is vertically integrated to increase the time of contact between the gas and medium. These three sections are repeated within one photobioreactor. Referring to FIG. 6, the photobioreactor is located in a water body (a) and contains an algae suspension (b) dispersed in a thin, even layer. The aeration section (e) is lowered under the surface of the water body (a) by a chamber (v). Chamber (v) contains a salt-in-water solution and has a higher density than the surrounding water body (a). Both external walls of aeration section (e) are formed by the lower reactor wall (c). Film (r) is attached to chamber (v) and separates the two walls. The connection is realized by punctual dots (s). At the upper end of the film (r) is connected to the upper reaction wall (d) without any interruption by welding a seam over the entire width of the reactor. The film (r) stretches over the entire aeration section (e).

In the degassing section (f), the lower (c) and upper (d) reactor walls are completely separated from each other, except for the connections at the boundary of the reactor, the connections near film (r), and the connections next to the growth section (l). The gas mixture leaving the liquid may be collected in, and removed from, the degassing section. A hose is fastened to one end of the degassing section (h) to lead the collected gas mixture out of the reactor.

The growth section (l) and the degassing section (f) are separated by two rows of connections (p) between the upper (d) and lower (c) reactor walls. The distance between the rows and the distance between connections in the same row is about 5 cm.

Tubular parallel chambers (i) are integrated in the aeration section (e) to supply a gas mixture comprising CO₂. The parallel chambers (t) are located near the middle of chamber (v) and at one side of film (r). The parallel chambers (i) lie below the degassing section (f). All parallel chambers (i) are connected to a long chamber (j) at one of the long sides of the reactor. Long chamber (j) runs over the entire length of the reactor and is 50 m in length. Chambers j) are manufactured by welding a PE film on the lower (c) reactor wall. Parallel chamber (i) has a width of about 1.5 cm and holes that about 1 cm from each other. Long chamber (j) has a width of about 2.5 cm and no holes.

When the as mixture is introduced into the reactor via parallel chambers (i), as bubbles rise in the aeration section (e). The rising bubbles carry algae medium along and the medium rises as well. New algae medium flows in through the other side of the aeration section (e) (in FIG. 6, right). The algae medium, which has risen in the left side of the aeration section (e) with the gas bubbles, moves to the degassing section (f) at the upper part of the aeration section (e). The film, which is added in the middle of the aeration section and is connected to the upper reactor wall in a watertight manner, guides the algae suspension into the degassing section (f) (in FIG. 6, left). Liquid and gaseous phases are separated further in the degassing section (f). More gas bubbles can escape from the liquid and can be led out of the reactor at end (h). The liquid is continuously pushed out of the aeration section in the direction of the growth section (l) by the flowing algae medium, powered by an energy source. The growth section (l) is partially separated from the degassing section (f) by connections between the upper (d) and lower (c) reactor walls. These connections prevent the invasion of larger quantities of air bubbles and thus prevent the formation of a large air cushion.

The photobioreactors of the present invention may be prepared at low cost, using readily available materials and with easy-to-apply processing methods. In addition, the photobioreactors of the present invention have high productivities and low susceptibility to contamination. By integrating the aeration and degassing sections, the photobioreactors of the present invention have a low energy consumption. By eliminating or minimizing the formation of large gas cushions, the wind susceptibility is reduced and making it, in principle, possible to lower the entire photobioreactor into the surrounding water body, e.g., as a protection against storms or for additional cooling. By increasing the time of contact between gas bubbles and the suspension, the photobioreactors have a good supply of CO₂, which, in turn results in a low energy input.

The photobioreactors of the present invention provide several advantages over the photobioreactors known in the art. For example, unlike photobioreactors having airlift pumps, the photobioreactors of the present invention integrate aeration directly into the photosynthetic part of the reactor and thus, they do not require hoses, tubes or fittings between the compartments for photosynthesis and for aeration. As a result of this integration, the aeration section and the photosynthetic section can be made from the same material, or the aeration section and the photosynthetic section can represent the same part of the reactor. Another advantage over photobioreactors having airlift pumps is that, in the photobioreactors of the present invention, the aeration and the degassing sections (as well as the optional additional sections) can be manufactured from flexible materials, e.g., thin plastic films.

The photobioreactors of the present invention provide advantages over known horizontal photobioreactors. Known horizontal photobioreactors supply CO₂ through an airlift pump (as described above), by diffusion or through injection at one or more locations. Diffusion cannot guarantee the mass transfer rates needed for a sufficient supply of CO₂ and removal of oxygen. And, in CO₂-injecting photobioreactors, the reactor must be positioned at an incline on a frame to ensure smooth operation. As a result. CO₂-injecting photobioreactors cannot be made from a flexible material. Because these reactors require a frame and cannot be made from a flexible material, their production costs are high.

### Photosynthetic or Mixotrophic Organism

The aeration systems and the photobioreactors of the present invention comprise a suspension of photosynthetic or mixotrophic organisms and growth media.

The aeration systems and the photobioreactors of the present invention are designed to be non-species dependent. The system settings, conformations, dimensions and contents may be adjusted to allow the growth of the selected photosynthetic or mixotrophic organism. Many species of photosynthetic and mixotrophic organisms have been discovered and characterized, and may be grown in the aeration systems and the photobioreactors of the present invention. Exemplary photosynthetic or mixotrophic organisms are vegetable tissues and monocellular organisms containing chloroplasts, photosynthetic bacteria and microalgae. The photosynthetic or mixotrophic organisms may be genetically modified to provide one or more desired characteristics for their culture, growth, harvesting or use. Methods for genetically modifying organisms are well known in the art and any such method may be used in the present invention.

Multiple species of photosynthetic or mixotrophic organisms may be grown within the aeration systems and the photobioreactors of the present invention. Each species may be present at all times, but the proportions may change depending on weather and environmental conditions.

The suspension contains growth media. Any growth media known in the an may be used and is preferably optimized for the selected species of photosynthetic or mixotrophic organism. Preferably, the growth medium comprises water, salt and nutrients.

The salinity of the suspension, concentration of cells, and pH may be regulated by any means known in the art. In some aspects of the invention, the suspension has a lower salinity than the water body. Preferably, the suspension has a salinity of about 1.8%. In some aspects of the invention, the suspension has a lower density than the surrounding water body. In some aspects of the invention, the average thickness of the medium for height) is about 5 cm.

The residence time of the suspension in each section of the aeration system and the photobioreactor is determined by the size and form of each section, the aeration rate, the fluid level and other factors known to those of skill in the art.

### Gas Mixture

A gas mixture is introduced into one of the aeration systems and photobioreactors of the present invention. The gas mixture may be comprised of a gas selected from the group consisting of nitrogen, ambient air, carbon dioxide, waste gases from industrial processes, combustion exhaust gases from stationary combustion chambers, power plant flue gases, waste gases from cement manufacture, waste gases from steel production, waste gases from ethanol production and any other selected gas source. The proportion, pressure and pre-treatment of the gases may be determined by the choice of organism being grown in the aeration system or photobioreactor. Preferably, the gas mixture comprises carbon dioxide or air having an increased carbon dioxide content. The carbon dioxide content in the air is in the range of 0 to 3.0% by volume, preferably of about 0.3% by volume.

The flow rate of the gas mixture within the aeration system or photobioreactor may be adjusted depending on the desired rate of photosynthesis, the CO₂ or oxygen levels, or energy consumption.

### Water Body

The photobioreactors are located in a water body. The water body may be sea water, brackish water, lagoon, pond, pool, lake, reservoir, including man-made water reservoir, or ocean. This water body may serve several purposes, including temperature regulation, structural support for the photobioreactor or light diffusion.

The water body has a salinity within the range of about 1% to about 25%. Preferably, the salinity is that of sea water, about 3.5%. More preferably, the salinity of the water body is greater than that of the suspension.

### Temperature Control

The water body aids the photobioreactor in maintaining a constant temperature range optimal for the species and strain of organism being grown in the photobioreactor, in general, the temperature of the organism suspension must be regulated to between about 5° to about: 50° C. for its own growth. The temperature within the photobioreactor may be regulated by any means known in the art. Suitable methods include, but are not limited to, those methods disclosed in U.S. Pat. No. 4,868,123 and U.S. Pat. No. 3,955,317.

The temperature may be controlled through the position of the photobioreactor in the water body. In particular, when the temperature of the suspension exceeds an upper reference temperature the photobioreactor may be immersed into the water body. Conversely, when the temperature of the suspension is below the minimum reference temperature, the photobioreactor may be raised in the water body.

### Methods

The present invention provides methods of growing photosynthetic or mixotrophic organisms. According to the method, a suspension comprising the organism is introduced into one of the photobioreactors of the present invention. The photobioreactor is located in a surrounding water body. The suspension is exposed to light and brought into contact with a gas mixture comprising CO₂ and other nutrients.

The present invention also provides methods of producing biomass. According to this method, a suspension comprising the photosynthetic or mixotrophic organisms is introduced into one of the photobioreactors of the present invention. The photobioreactor is located in a surrounding water body. The organisms are grown in a suspension in the photobioreactor. The suspension is exposed to light and brought into contact with a gas mixture comprising CO₂ and other nutrients. The organisms produce a biomass, which is then harvested. The biomass may be harvested by methods known in the art.

The present invention also provides methods of producing a biofuel. According to this method, a suspension comprising the photosynthetic or mixotrophic organisms is introduced into one of the photobioreactors of the present invention. The photobioreactor is located in a surrounding water body. The organisms are grown in a suspension in the photobioreactor. The suspension is exposed to tight and brought into contact with a gas mixture comprising CO₂ and other nutrients. The organisms produce a biomass, which is then harvested. Lipids, carbohydrates, proteins, vitamins, antioxidants, components from the photosynthetic or mixotrophic organism, and other components from the biomass are convened into biofuel. The conversion may be performed by methods known in the art.

The present invention also provides methods of producing a product selected from the group consisting of biochemicals, amino acids, fine chemicals, nutriceuticals, pharmaceuticals, energy products (ethanol, methane, hydrogen, fatty adds, fats and other lipids, highly energetic compound, propanol, butanol, gasoline-like fuel, diesel-like fuel, alkanes. Aeries, alcohols, organic acids, aromatic compounds), protein, feed for cattle or other species, fish feed, including feed for fish larvae and teed for other potential aquaculture uses, e.g., food for shrimps, crabs, oysters and their larvae, protein source for human nutrition and mineral rich food for human consumption. According to this method, a suspension comprising the photosynthetic or mixotrophic organisms is introduced into one of the photobioreactors of the present invention. The photobioreactor is located in a surrounding water body. The organisms are grown in a suspension in the photobioreactor. The suspension is exposed to light and brought into contact with a gas mixture comprising CO₂ and other nutrients. The organisms produce a biomass, which is then harvested. Lipids, carbohydrates, proteins, vitamins, antioxidants, components from the photosynthetic or mixotrophic organism, and other components from the biomass are converted into the desired product. The conversion may be performed by methods known in the art.

While particular materials, formulations, operational sequences, process parameters, and end products have been set forth to describe and exemplify this invention, they are not intended to be limiting. Rather, it should be noted by those ordinarily skilled in the art that the written disclosures are exemplary only and that various other alternatives, adaptations, and modifications may be made within the scope of the present invention. Accordingly, the present invention is not limited to the specific embodiments illustrated herein, but is limited only by the following claims.

1. An aeration system comprising:
(a) an aeration section, and
(b) a degassing section,
wherein the walls of the aeration system are made from a flexible material; and
wherein connections between the upper and lower walls form the aeration section and the degassing section.

2. The aeration system according to claim 1, wherein the flexible material is polyethylene.

3. The aeration system according to claim 1, wherein the aeration section comprises parallel chambers and a long chamber,
wherein the chambers supply a gas mixture to the aeration section.

4. The aeration system according to claim 1, wherein the degassing section comprises connections to periphery to transport excess gas out of the aeration system.

5. The aeration system according to claim 1, further comprising a growth section.

6. The aeration system according to claim 1, further comprising a photosynthetic or mixotrophic organism and growth medium.

7. A horizontal photobioreactor comprising an aeration system according to claim 1.

8. A horizontal photobioreactor comprising an aeration system, wherein the aeration system comprises:
(a) an aeration section, and
(b) a degassing section,
wherein connections between the upper and lower walls of the photobioreactor form the aeration section and the degassing section.

9. The horizontal photobioreactor according to claim 8, wherein, in the aeration section, the upper and lower walls are directly connected.

10. The horizontal photobioreactor according to claim 8, further comprising a growth section.

11. A horizontal photobioreactor comprising an aeration system, wherein the aeration system comprises:
(a) an aeration section, and
(b) a degassing section,
wherein the aeration section is lowered below the degassing section.

12. A horizontal photobioreactor comprising an aeration system, wherein the aeration system comprises:
(a) an aeration section,
(b) a degassing section, and
(c) a growth section,
wherein the aeration section is lowered below the degassing section and the growth section;
and the upper and lower walls of the photobioreactor are connected to form the degassing section and the growth section.

13. A horizontal photobioreactor comprising an aeration system, wherein the aeration system comprises:
(a) an aeration section,
(b) a degassing section, and
(c) a growth section,
wherein the aeration section, degassing section and the growth section are lowered below a surrounding water body.

14. The horizontal photobioreactor according to any one of claims 11-13, wherein the section is lowered by a method selected from the group consisting of manufacturing the photobioreactor wall using a material with high density, increasing the amount of material in the photobioreactor wall, fastening the section to an object outside of the photobioreactor, adding a material with higher density than a surrounding water body to the section, and manufacturing the section to contain one or more chambers that can be filled with a high density material.

15. The horizontal photobioreactor according to any one of claims 8 and 10-13, wherein the upper wall and the lower wall comprise a flexible material.

16. The horizontal photobioreactor according to claim 15, wherein the flexible material is polyethylene.

17. The horizontal photobioreactor according to any one of claims 8 and 10-13, wherein the aeration section comprises parallel chambers and a long chamber,
wherein the chambers supply a gas mixture to the aeration section.

18. The horizontal photobioreactor according to claim 17, wherein the gas mixture comprises carbon dioxide.

19. The horizontal photobioreactor according to any one of claims 8 and 10-13, wherein the degassing section comprises connections to periphery to transport excess gas out of the aeration system.

20. The horizontal photobioreactor according to any one of claims 8 and 10-13, further comprising a photosynthetic or mixotrophic organism and growth medium.

21. The horizontal photobioreactor according to claim 10, further comprising a film where the aeration section and growth section meet, wherein the film and the lower wall of the photobioreactor are connected.

22. The horizontal photobioreactor according to claim 10, wherein the growth section and the degassing section are separated by connections between the upper and lower walls of the photobioreactor.

23. The horizontal photobioreactor according to any one of claims 8 and 10-13, wherein the sections are repeated within the photobioreactor.

24. A system of horizontal photobioreactors comprising two or more photobioreactors connected to each other,
wherein at least one of the photobioreactors is as defined in any one of claims 7, 8 and 10-13.

25. The system according to claim 24, wherein the photobioreactors are arranged in a circular pattern.

26. A method of growing a photosynthetic or mixotrophic organism comprising:
(a) introducing a suspension comprising the photosynthetic or mixotrophic organism and growth medium into the photobioreactor of any one of claims 7, 8 and 10-13, wherein the photobioreactor is located in a surrounding water body;
(b) exposing the suspension to light; and
(c) contacting the suspension with a gas mixture comprising CO₂.

27. A method of producing a biomass comprising:
(a) growing a photosynthetic or mixotrophic organism in a growth medium in the photobioreactor of any of claims 7, 8 and 10-13, wherein the photobioreactor is surrounded by a water body;
(b) harvesting the biomass.

28. A method of producing a biofuel comprising:
(a) growing a photosynthetic or mixotrophic organism in a growth medium in the photobioreactor of any one of claims 7, 8 and 10-13, wherein the photobioreactor is surrounded by a water body;
(b) harvesting the organism; and
(c) converting one or more selected from the group consisting of lipids, carbohydrates, proteins, vitamins, or antioxidants from the organism, and components of the organism into the biofuel.

29. A method for producing a product comprising:
(a) growing a photosynthetic or mixotrophic organism in a growth medium in the photobioreactor of any one of claims 7, 8 and 10-13, wherein the photobioreactor is surrounded by a water body;
(b) harvesting the organism; and
(c) converting one or more selected from the group consisting of lipids, carbohydrates, proteins, vitamins, or antioxidants from the organism and components of the organism into the product,
wherein the product is selected from the group consisting of biochemicals, amino acids, fine chemicals, nutriceuticals, pharmaceuticals, energy products, protein, feed for cattle or other species, fish feed, protein source for human nutrition and mineral rich food for human consumption.
